(19) United States
(12) Patent Application Publication
(10) Pub. No.: US 2025/0249156 A1
(43) Pub. Date: Aug. 7, 2025

(54) BREAST-PUMPING CARE ACCESSORY AND CARE CONTROLLING METHOD

(71) Applicant: SHENZHEN ZXD TRADING CO., LTD, Shenzhen (CN)

(72) Inventor: Wei Yi, Shenzhen (CN)

(21) Appl. No.: 18/648,411

(22) Filed: Apr. 28, 2024

(30) Foreign Application Priority Data
Feb. 5, 2024 (CN) 202410171668.6
Feb. 5, 2024 (CN) 202410173306.0

(51) Int. Cl. A61M 1/06; A61F 7/00; A61F 7/02; A61N 1/04; A61N 1/08; A61N 1/20

(52) U.S. Cl. CPC A61M 1/069; A61F 7/007; A61M 1/062; A61N 1/0484; A61N 1/08; A61N 1/205; A61F 2007/0021; A61F 2007/0093; A61F 2007/0096; A61F 2007/0228; A61M 2205/054; A61M 2205/581

(57) ABSTRACT

A breast-pumping care accessory and a care method are disclosed. The accessory includes a first attaching element, configured to be attached to a breast of a living body when in operation; a second attaching element; a heating element, located between the first attaching element and the second attaching element; a controller, electrically connected to the heating element; and an energy supply element, configured to supply electric energy to the heating element and the controller. The controller is configured to perform a care controlling method including: obtaining a target breast-pumping care mode; obtaining a heating curve based on the target breast-pumping care mode from a curve library and taking the heating curve as a target curve; and controlling the heating element to generate heat based on the target curve. The target breast-pumping care mode includes any one of a pre-pumping care mode, an in-pumping care mode, and a post-pumping care mode.

### CROSS REFERENCE TO RELATED APPLICATIONS

This application claims priority to Chinese Patent Application Nos. 202410171668.6 and 202410173306.0, filed on Feb. 5, 2024, the contents of which are herein incorporated by reference in their entirety.

### TECHNICAL FIELD

The present disclosure relates to the technical field of maternal and infant equipment, in particular to a breast-pumping care accessory and a care controlling method for the breast-pumping care accessory.

### BACKGROUND

Breastmilk is the most natural, safest, and most complete natural food for infant growth, which makes breast-pumping care particularly important. For existing breast pumps, selected strength is used to pump or suck out milk, but the breast-pumping care is ignored, which results in poor breast health and low breastmilk production.

### SUMMARY

In one aspect, a breast-pumping care accessory is provided. The breast-pumping care accessory comprises a care assembly. The care assembly comprises: a first attaching element, configured to be attached to a breast of a living body when the breast-pumping care accessory is in operation; a second attaching element; a heating element, located between the first attaching element and the second attaching element, the first attaching element being attached to the second attaching element to fix the heating element; a controller, electrically connected to the heating element; and an energy supply element, configured to supply electric energy to the heating element and the controller. The controller is configured to perform a care controlling method, and the method comprises: obtaining a target breast-pumping care mode; obtaining a heating curve, based on the target breast-pumping care mode, from a curve library, and taking the heating curve as a target curve; and controlling the heating element to generate heat based on the target curve. The target breast-pumping care mode comprises any one of a pre-pumping care mode, an in-pumping care mode, and a post-pumping care mode.

In some embodiments, the second attaching element comprises a mounting portion and a fixing portion, the mounting portion is connected to the fixing portion, the mounting portion is configured to mount the heating element, and the fixing portion is configured to fix the care assembly on a breast pump.

In some embodiments, the breast-pumping care accessory further comprises a wearable strap. The wearable strap is connected to an upper end and a lower end of the care assembly, or the wearable strap is connected to a left end and a right end of the care assembly; wherein when the breast-pumping care accessory is in operation, the wearable strap is configured to fix the care assembly to the breast of the living body, such that the first attaching element capable of being attached to the breast of the living body.

In some embodiments, the care assembly is provided with at least one ventilation through hole; the at least one ventilation through hole penetrates the first attaching element and the second attaching element, or the at least one ventilation through hole penetrates the first attaching element, the heating element, and the second attaching element.

In some embodiments, the first attaching element and the second attaching element are made of elastic silicon; the heating element comprises a flexible membrane and a printed circuit, the printed circuit is located on the flexible membrane, and the controller is electrically connected to the printed circuit. The flexible membrane comprises a plurality of flexible pieces, each of the plurality of flexible pieces is fan-shaped and comprises a first arc portion and a second arc portion, an arc length of the first arc portion is less than an arc length of the second arc portion, the plurality of flexible pieces are arranged in a ring and distributed at intervals, and the first arc portions of every adjacent two of the plurality of flexible pieces are connected to each other. When the breast-pumping care accessory is in operation, the first arc portion is located between a nipple of the breast of the living body and the second arc portion.

In some embodiments, the heating element further comprises one or more contact electrodes, the one or more contact electrodes are electrically connected to the printed circuit, the first attaching element defines one or more electrode through holes, the one or more contact electrodes and the one or more electrode through holes are in one-to-one correspondence, the one or more contact electrodes pass through the one or more electrode through holes correspondingly, and the one or more contact electrodes are configured to provide microcurrent stimulation to the breast of the living body.

In some embodiments, when the care control method is executed, the controller is further configured to implement operations comprising: determining an electrode control configuration based on the target breast-pumping care mode; and enabling each of the one or more contact electrodes to generate a microcurrent based on the electrode control configuration.

In some embodiments, the care assembly further comprises a first temperature sensor, the first temperature sensor is arranged on the first attaching element, and the first temperature sensor is configured to detect a temperature of the breast of the living body, and the controller is electrically connected to the first temperature sensor. When the care control method is executed, the controller is further configured to implement operations comprising: obtaining the temperatures of the breast detected by the first temperature sensor; generating a breast-temperature curve based on the temperatures of the breast, obtaining an accumulative heating amount based on the breast-temperature curve by using the calculus method; and enabling each of the one or more contact electrodes to generate the microcurrent based on the electrode control configuration and the accumulative heating amount.

In some embodiments, the care assembly further comprises a second temperature sensor, the second temperature sensor is configured to detect an ambient temperature of the living body, and the controller is electrically connected to the second temperature sensor. When the care controlling method is executed, in the operation of controlling the heating element to generate heat base on the target curve, the controller is further configured to implement operations comprising: obtaining the ambient temperature detected by the second temperature sensor; correcting the target curve based on the ambient temperature; and enabling the heating element to generate heat based on the corrected target curve.

In some embodiments, the breast breast-pumping care accessory further comprises a sound reminder, and the controller is electrically connected to the sound reminder. When the care controlling method is executed, after the operation of controlling the heating element to generate heat based on the target curve, the controller is further configured to implement operations comprising: obtaining a heating completion signal; and in response to the heating completion signal, controlling the sound reminder to play a prompt sound to remind the completion of the care.

In another aspect, a care controlling method for a breast-pumping care accessory is provided. The breast-pumping care accessory comprises: a first attaching element, configured to be attached to a breast of a living body when the breast-pumping care accessory is in operation; a second attaching element; a heating element, located between the first attaching element and the second attaching element, the first attaching element being attached to the second attaching element to fix the heating element; a controller, electrically connected to the heating element and configured to implement the care controlling method; and an energy supply element, configured to supply electric energy to the heating element and the controller. The care controlling method comprises: obtaining a target breast-pumping care mode; obtaining a heating curve, based on the target breast-pumping care mode, from a curve library, and taking the heating curve as a target curve; and controlling the heating element to generate heat based on the target curve. The target breast-pumping care mode comprises any one of a pre-pumping care mode, an in-pumping care mode, and a post-pumping care mode.

### BRIEF DESCRIPTION OF THE DRAWINGS

In order to more clearly illustrate the technical solutions in the embodiments of the present disclosure or the related art, the accompanying drawings required to be used in the description of the embodiments of the present disclosure or the related art will be briefly introduced below. Apparently, the accompanying drawings in the following description are only some embodiments of the present disclosure, and for those skilled in the art, other drawings may also be obtained based on these drawings without paying creative works.

FIG. 1 is a structural block diagram of a breast-pumping care accessory according to some embodiments of the present disclosure.

FIG. 2 is a flow chart of a care controlling method according to some embodiments of the present disclosure.

FIG. 3 is a partial structural schematic view of a breast-pumping care accessory according to some embodiments of the present disclosure.

FIG. 4 is a partial structural schematic view of a breast-pumping care accessory according to some embodiments of the present disclosure.

FIG. 5 is a schematic diagram illustrating a heating curve according to some embodiments of the present disclosure.

FIG. 6 is a schematic diagram illustrating a heating curve according to some embodiments of the present disclosure.

FIG. 7 is a schematic diagram illustrating a heating curve according to some embodiments of the present disclosure.

FIG. 8 is a flow chart of a care controlling method according to some embodiments of the present disclosure.

FIG. 9 is a flow chart of a care controlling method according to some embodiments of the present disclosure.

FIG. 10 is a partial structural schematic view of a breast-pumping care accessory according to some embodiments of the present disclosure.

FIG. 11 is a partial structural schematic view of a breast-pumping care accessory according to some embodiments of the present disclosure.

FIG. 12 is a structural schematic view of a breast-pumping care accessory according to some embodiments of the present disclosure.

### DETAILED DESCRIPTION

The technical solutions in the embodiments of the present disclosure will be clearly and completely described below in conjunction with the accompanying drawings in the embodiments of the present disclosure. Apparently, the embodiments described are part of the embodiments of the present disclosure, not all embodiments. Based on the embodiments in the present disclosure, those skilled in the art may obtain other embodiments without paying any creative work. All these embodiments fall within the scope of protection of the present disclosure.

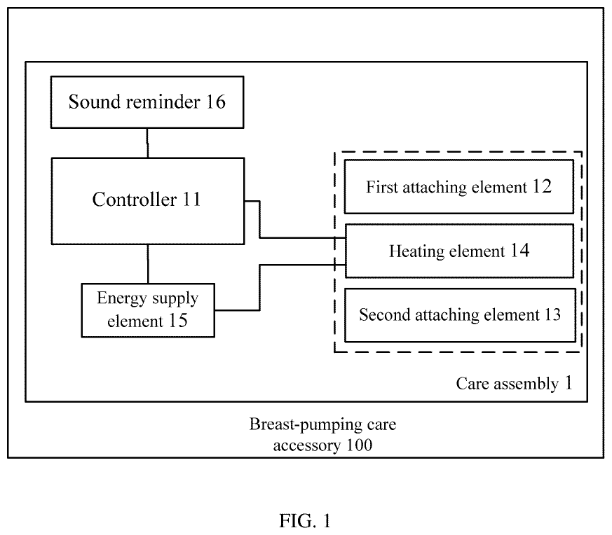

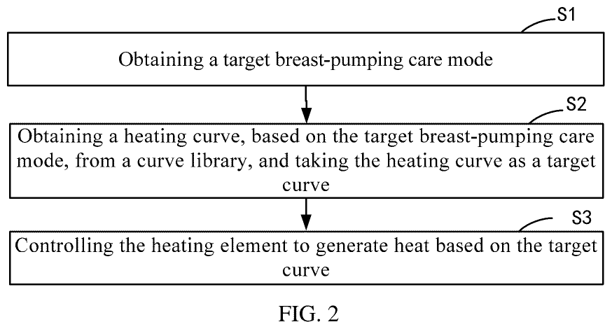

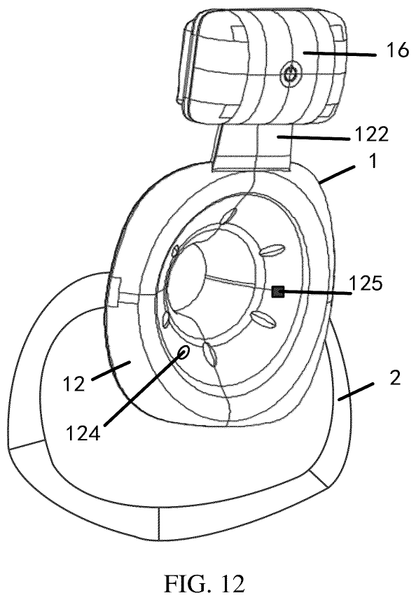

As shown in FIGS. 1-4 and FIGS. 10-12, some embodiments of the present disclosure provide a breast-pumping care accessory 100. The breast-pumping care accessory 100 includes a care assembly 1. The care assembly 1 includes the following components including a first attaching element 12, a second attaching element 13, a heating element 14, a controller 11, and an energy supply element 15.

The first attaching element 12 is configured to be attached to a breast of a living body when the breast-pumping care accessory 100 is in operation.

The heating element 14 is located between the first attaching element 12 and the second attaching element 13. The first attaching element 12 is attached to the second attaching element 13 to fix the heating element 14. That is to say, the heating element 14 is sandwiched between the first attaching element 12 and the second attaching element 13.

The controller 11 is electrically connected to the heating element 14.

The energy supply element 15 is configured to supply electric energy to the heating element 14 and the controller 11.

The controller 11 is configured to perform a care controlling method. The following operations may be executed when the care controlling method is performed.

S1, a target breast-pumping care mode is obtained.

S2, a heating curve is obtained, based on the target breast-pumping care mode, from a curve library and used or taken as a target curve.

S3, the heating element 14 is controlled or enabled to generate heat based on the target curve.

In some embodiments, the target breast-pumping care mode includes any one of a pre-pumping care mode, an in-pumping care mode, and a post-pumping care mode.

In some embodiments, the breast-pumping care is achieved by heating the breast, the heating has a hot compress effect, and the hot compress may promote the blood circulation of the breast to promote milk secretion, which is conducive to the breast health and increases the amount of milk sucked out. Based on the target breast-pumping care mode, the heating curve is obtained from the curve library and taken as the target curve. Based on the target curve, the heating element 14 is controlled or enabled to generate heat, the breast is heated by adopting the heating curve corresponding to or matching the target breast-pumping care mode. In this way, individual demands or personalized demands of different target breast-pumping care modes for heating may be satisfied. Therefore, the hot compress effect is improved, which is conducive to the breast health, and the amount of milk sucked out may be further increased.

It will be appreciated that, the breast-pumping care accessory 100 in some embodiments of the present disclosure may be manufactured and sold as an independent device or used as an accessory for a breast pump in factory.

In some embodiments, the first attaching element 12 may be implemented as a flange cover. It will be appreciated that, the first attaching element 12 may also be in other shapes, such as a circular ring, a sector, etc., which is not limited here.

In some embodiments, the first attaching element 12 may be made of silicone, thereby facilitating a better attachment to the breast of the living body. It will be appreciated that, the first attaching element 12 may also be made of other materials, such as plastic, latex, etc., which is not limited here.

In some embodiments, the second attaching element 13 may be implemented as a flange cover. It will be appreciated that, the second attaching element 13 may also be in other shapes, such as a circular ring, a sector, etc., which is not limited here.

In some embodiments, the second attaching element 13 may be made of silicone, thereby facilitating a better attachment to the heating element 14 and the first attaching element 12. It will be appreciated that, the second attaching element 13 may also be made of other materials, such as plastic, latex, etc., which is not limited here.

The energy supply element 15 is electrically connected to the heating element 14 and the controller 11, and is configured to supply electric energy or power to the heating element 14 and the controller 11. The energy supply element 15 may be a power supply device that adopts a rechargeable battery, and may also be a power supply device that adopts an adapter. The structure of the energy supply element 15 may be selected from the related art, and is not repeated here.

In some embodiments, the energy supply element 15 may provide a voltage of 5-10V.

The heating element 14 is an element that is configured to convert the electrical energy provided by the energy supply element 15 into thermal energy. For example, the heating element 14 includes an electrical-to-thermal energy sub-component and a heat-conduction sub-component. The electrical-to-thermal energy sub-component is a component configured to convert the electrical energy provided by the energy supply element 15 into the thermal energy. The heat-conduction sub-component is a component configured to diffuse or radiate the thermal energy generated by the electrical-to-thermal energy sub-component. The thermal energy is transmitted to the breast of the living body through the first attaching element 12, such that the hot compress of the breast of the living body is realized.

In some embodiments, the maximum heating temperature of the heating element 14 is less than or equal to 65° C., thereby avoiding scalding the breast. It will be appreciated that when the thickness of the first attaching element 12 is relatively large, the maximum heating temperature of the heating element 14 may also be greater than 65° C., as long as it is possible to ensure that the thermal energy transmitted to the breast of the living body through the first attaching element 12 will not scald the breast.

The controller 11 includes a non-transitory computer-readable memory and a processor. A computer program is stored in the non-transitory computer-readable memory and may run on the processor. A care controlling method may be performed when the processor executes the computer program.

The controller 11 may include a mounting box 16. The non-transitory computer-readable memory and the processor are independently configured in the mounting box 16, and the mounting box 16 may be connected to the first attaching element 12 and/or the second attaching element 13. The mounting box 16 may also be arranged independently of the first attaching element 12 and the second attaching element 13. When the mounting box 16 is arranged independently of the first attaching element 12 and the second attaching element 13, the controller 11 is connected to the heating element 14 through a connecting wire.

The energy supply element 15 may be arranged in the mounting box 16, and the energy supply element 15 is connected to the heating element 14 and the controller 11 through connecting wires.

In the pre-pumping care mode, the care for the breast is performed before pumping. In the in-pumping care mode, the care for the breast is performed during pumping. In the post-pumping care mode, the care is performed for the breast after pumping.

For the operation S1, the target breast-pumping care mode is obtained through the following methods: obtaining a breast-pumping care mode input by the user in real time, and taking the input breast-pumping care mode as the target breast-pumping care mode; or obtaining a pre-stored care mode and taking the pre-stored care mode as the target breast-pumping care mode; or obtaining the care mode set by default at the factory and taking the default care mode as the breast-pumping care mode.

In some embodiments, the target breast-pumping care mode is obtained through the following operations. A care instruction input by the user may be obtained. In case that the care instruction carries a care mode, the carried care mode is taken as the target breast-pumping care mode. In case that the care instruction does not carry a care mode, when a care mode is stored in the memory, the care mode stored in the memory is taken as the target breast-pumping care mode. In case that there is no care mode stored in the memory, the care mode set by default at the factory is taken as the target breast-pumping care mode. In some embodiments, the user may input the care instruction through a button or touch screen on the breast-pumping care accessory 100. In some embodiments, the user may also input the care instruction through a mobile device, and the mobile device further sends the care instruction to the breast-pumping care accessory 100 through a network connection.

For the operation S2, the curve library is configured to describe or represent the correspondence or mapping relationship between the care mode and the heating curve. The target breast-pumping care mode may be searched or looked up among care modes stored in the curve library. The heating curve corresponding to the searched or found care mode in the curve library is taken as the target curve. That is, the heating curve corresponding to the target breast-pumping care mode may be searched or looked up in the curve library.

The heating curve is a heating curve that is calibrated based on the care requirements of the target breast-pumping care mode. The horizontal coordinate of the heating curve is the time point, and the vertical ordinate of the heating curve is the heating temperature.

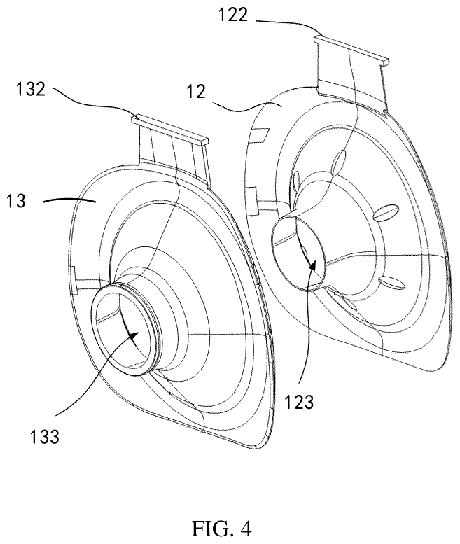

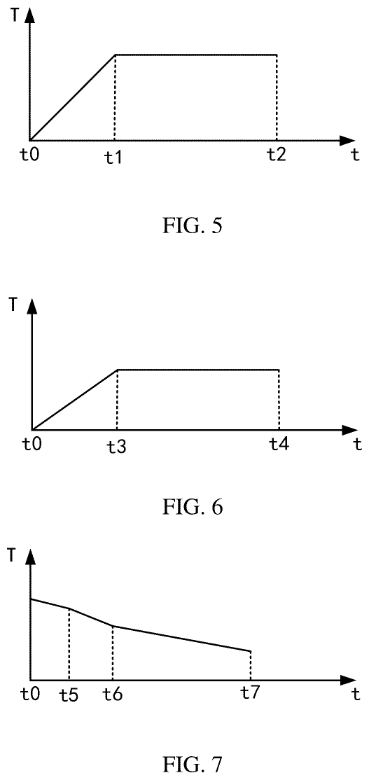

In case that the target breast-pumping care mode is the pre-pumping care mode, as shown in FIG. 5, the heating curve corresponding to the pre-pumping care mode is successively divided into a first temperature-rising or warming section (t0-t1) and a first constant-temperature section (t1-t2), thereby realizing the heating from a temperature-rising pre-heating stage (t0-t1) to a constant-temperature stage (t1-t2). Using the transition from the temperature-rising pre-heating stage to the constant-temperature stage is conducive to gradually promoting the blood circulation of the breast, and also avoiding a high temperature at the beginning, thereby improving the comfort of the hot compress.

In case that the target breast-pumping care mode is the in-pumping care mode, as shown in FIG. 6, the heating curve corresponding to the in-pumping care mode is successively divided into a second temperature-rising or warming section (t0-t3) and a second constant-temperature section (t3-t4). A temperature at t1 shown in FIG. 5 is greater than a temperature at t3 shown in FIG. 6, thereby realizing the heating from the temperature-rising pre-heating stage (t0-t3) to the constant-temperature stage (t3-t4). Using the transition from the temperature-rising pre-heating stage to the constant-temperature stage is conducive to gradually promoting the blood circulation of the breast, and also avoiding a high temperature at the beginning, thereby improving the comfort of the hot compress. The breast is expressed during the pumping process, which leads to an acceleration in the blood circulation. Besides, the blood circulation may be further promoted by heating. Therefore, the blood circulation promoted by heating and the blood circulation accelerated by the breast being expressed during the pumping process may have a double combination result. The hot express after the double combination result may have the best blood circulation effect in case that the temperature at t1 is greater than the temperature at t3.

In case that the target breast-pumping care mode is the post-pumping care mode, as shown in FIG. 7, the heating curve corresponding to the post-pumping care mode is successively divided into multiple heating sections with a decreasing temperature (FIG. 7 illustrates three heating sections with the decreasing temperatures, the three heating sections are t0-t5, t5-t6, and t6-t7). In this way, it is possible to gradually reduce or decrease the heating temperature, such that the temperature of the heating element 14 is closer to the temperature of the human body when the care is completed. The heating temperature is gradually decreased or reduced, which is conducive to gradually promoting the blood circulation of the breast. Besides, the temperature of the heating element 14 is closer to the temperature of the human body when the care is completed, thereby reducing the temperature difference between the breast and the ambient environment when the first attaching element 12 is removed from the breast of the living body after the hot compress, improving the comfort level, and in turn improving the user experience.

In some embodiments, the heating curve for the post-pumping care mode may be a combination of one or more heating sections with decreasing temperatures and one or more heating sections with a constant temperature.

The heating sections with decreasing temperatures refer to the sections with heating temperatures at the beginning of the heating sections greater than the heating temperatures at the end of the heating sections.

It will be appreciated that, although each section shown in FIGS. 5-7 is a straight line, however, in practical applications, the section at each stage of the heating curve according to some embodiments of the present disclosure may be straight or curved, which is not limited here.

In some embodiments, the second attaching element 13 includes a mounting portion 134 and a fixing portion 135. The mounting portion 134 is connected to the fixing portion 135. The mounting portion 134 is configured to mount the heating element 14. The fixing portion 135 is configured to fix the care assembly 1 on the breast pump. By means of the fixing portion 135, the care assembly 1 may be quickly fixed to the breast pump, such that it is possible to provide care to the breast during pumping.

In some embodiments, the mounting portion 134 may be implemented as a flange cover. It will be appreciated that, the mounting portion 134 may also be in other shapes, may also be in other shapes, such as a circular ring, a sector, etc., which is not limited here.

In some embodiments, the mounting portion 134 may be made of silicone, thereby facilitating a better attachment to the heating element 14 and the first attaching element 12. It will be appreciated that, the mounting portion 134 may also be made of other materials, such as plastic, latex, which is not limited here.

In some embodiments, the fixing portion 135 may be made of elastic material. An assembly of the mounting portion 134, the heating element 14, and the first attaching element 12 are put into a suction mechanism of the breast pump. The breast of the living body is heated through the assembly. The breast of the living body is expressed through the breast pump to express milk or suck the milk out. The fixing portion 135 is sleeved on the breast pump. The suction mechanism includes a storage and a suction cover. The suction cover is arranged on the storage. The assembly of the mounting portion 134, the heating element 14, and the first attaching element 12 is put into the suction cover. The fixing portion 135 is sleeved on a periphery of the storage.

It will be appreciated that, the fixing portion 135 may also be arranged in other ways, which is not limited here.

In some embodiments, the breast-pumping care accessory 100 further includes a wearable strap or band 2. The wearable strap 2 is connected to both an upper end and a lower end of the care assembly 1. Or, the wearable strap 2 is connected to both a left end and a right end of the care assembly 1. When the breast-pumping care accessory 100 is in operation, the wearable strap 2 is configured to fix the care assembly 1 to the breast of the living body, such that the first attaching element 12 is attached to the breast of the living body. The care assembly 1 is fixed to the breast of the living body through the wearable strap 2, such that the care assembly 1 does not need to be held by the hand during the whole care process, and the user experience is improved.

The wearable strap 2 may be in shape of a strap or a band. The shape of the wearable strap 2 is not limited here. In some embodiments, the breast pump may be mounted on the living body by means of the wearable strap 2 and the care assembly 1.

In some embodiments, the care assembly 1 defines at least one ventilation through hole 126. The ventilation through hole 126 penetrates both the first attaching element 12 and the second attaching element 13. Or, the ventilation through hole 126 penetrates the first attaching element 12, the heating element 14, and the second attaching element 13. When the breast-pumping care accessory 100 is in operation, part of the skin of the breast may directly communicate with the outside through the ventilation through hole 126, and it is possible to reduce the stuffiness.

In a region where the first attaching element 12, the heating element 14, and the second attaching element 13 exist simultaneously, the ventilation through hole 126 penetrates the first attaching element 12, the heating element 14, and the second attaching element 13. In a region where only the first attaching element 12 and the second attaching element 13 exist, the ventilation through hole 126 penetrates both the first attaching element 12 and the second attaching element 13.

In some embodiments, both the first attaching element 12 and the second attaching element 13 may be made of elastic silicon.

The heating element 14 may include a flexible membrane 145 and a printed circuit 146. The printed circuit 146 is located on the flexible membrane 145, and the controller 11 is electrically connected to the printed circuit 146.

The flexible membrane 145 has a plurality of flexible pieces 1451. Each of the plurality of flexible pieces may be sector-shaped and may have a first arc portion 142 and a second arc portion 141. An arc length of the first arc portion 142 is less than an arc length of the second arc portion 141. The plurality of flexible pieces are arranged in a circular ring and distributed at intervals. The first arc portions 142 of the every adjacent two of the flexible pieces 1451 are connected to each other. Besides, the second arc portions 141 of the every adjacent two of the flexible pieces 1451 are spaced apart from each other, such that a gap is defined between the second arc portions 141 of the every adjacent two of the flexible pieces 1451. When the breast breast-pumping care accessory 100 is in operation, the first arc portions 142 are located between the nipple of the breast of the living body and the second arc portions 141. By forming the flexible membrane 145 with the plurality of flexible pieces 1451 and having the second arc portions 141 being freely arranged, the second arc portions 141 of the plurality of flexible pieces 1451 have certain movement space, such that when the care assembly 1 is expressed and deformed, the heating element 14 is not easy to be damaged, and the service life of the care assembly 1 is prolonged.

It will be appreciated that, in some embodiments of the present disclosure, the flexible membrane 145 may be in a shape similar to a cone, such that the heating element 14 is more suitable for or fitted to the breast of the living body, and is further conducive to improving the effect of the hot compress.

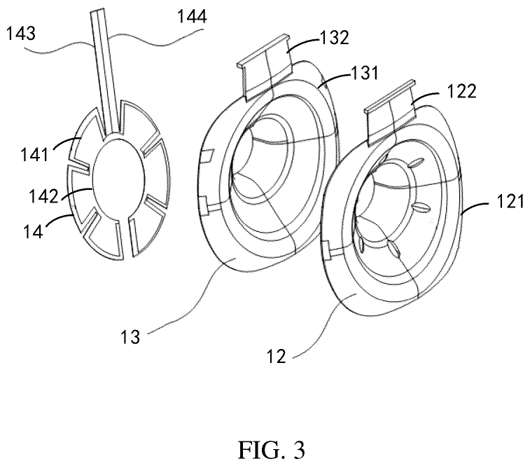

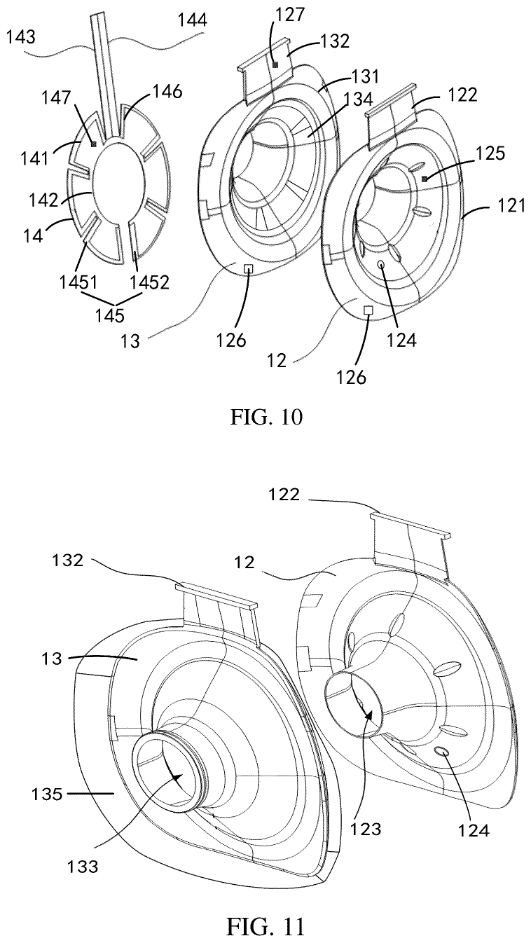

As shown in FIG. 3 and FIG. 4, the flexible membrane 145 of the heating element 14 includes six flexible pieces 1451 (in practical applications, as shown in FIG. 10, the flexible membrane 145 of the heating element 14 may include at least two flexible pieces). The first arc portions 142 of every adjacent two flexible pieces 1451 are connected to each other. Reference numeral 141 refers to the second arc portion. A positive connecting wire 143 is electrically connected to the printed circuit 146 on the flexible membrane 145. A negative connecting wire 144 is electrically connected to the printed circuit 146 on the flexible membrane 145. When the breast breast-pumping care accessory 100 is in operation, a first mounting cover 121 of the first attaching element 12 may be attached to the breast of the living body, the flexible membrane 145 and the printed circuit 146 are arranged between the first mounting cover 121 of the first attaching element 12 and a second mounting cover 131 of the second attaching element 13. A first connecting portion 122 extends from the first attaching element 12, and a second connecting portion 132 extends from the second attaching element 13. The positive connecting wire 143 and the negative connecting wire 144 are located between the first connecting portion 122 and the second connecting portion 132. The first connecting portion 122 and the second connecting portion 132 are all connected to the controller 11. A first channel 123 of the first attaching element 12 and a second channel 133 of the second attaching element 13 cooperatively form a milk discharge channel.

In some embodiments, the heating element 14 further includes one or more contact electrodes 147. The one or more contact electrodes 147 are electrically connected to the printed circuit 146. The first attaching element 12 defines one or more electrode through holes 124. The one or more contact electrodes 147 and the one or more electrode through holes 124 are in one-to-one correspondence. The one or more contact electrodes 147 pass through the one or more electrode through holes 124 correspondingly. The one or more contact electrodes 147 are configured to provide microcurrent stimulation to the breast of the living body. The mammary glands may be massaged and dredged through the microcurrent stimulation, which promotes the blood circulation in the breast and further promotes the milk secretion.

The one or more contact electrodes 147 pass through the first attaching element 12 and are in direct contact with the breast of the living body. The breast of the living body is stimulated by the microcurrent stimulation.

It will be appreciated that, the heating element 14 includes at least one contact electrode 147. The at least one contact electrode 147 may be distributed based on the distribution of the mammary glands of the breast of the living body, such that each contact electrode 147 may directly contact with the skin corresponding to the mammary glands, thereby improving the massage effect.

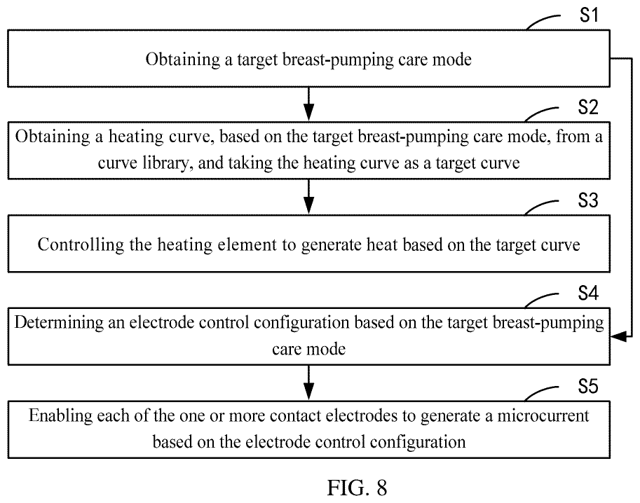

As shown in FIG. 8, in case that the heating element 14 includes the one or more contact electrodes 147, in some embodiments, when the care control method is executed, the controller 11 is further configured to implement the following operations.

S4, an electrode control configuration is determined based on the target breast-pumping care mode.

S5, each of the one or more contact electrodes 147 may be controlled or enabled to generate a microcurrent based on the electrode control configuration.

In some embodiments, the electrode control configuration is determined based on the target breast-pumping care mode, each of the one or more contact electrodes 147 may be controlled or enabled to generate a microcurrent based on the electrode control configuration. By using the electrode control configuration corresponding to or matching the target breast-pumping care mode to perform the microcurrent massage on the breast, it is possible to satisfy the individual demands of different target breast-pumping care modes for massage, which improves the massage effect, is conductive to the health of the mammary glands of the breast, and further increases the amount of milk sucked out.

For the operation S4, based on the target breast-pumping care mode, a look-up table method is employed to determine the electrode control configuration.

In some embodiments, the electrode control configuration describes the correspondence among a generation time point of the microcurrent, a duration of a single massage, and a standard amount of current.

It will be appreciated that, all the contact electrodes 147 in the breast-pumping care accessory 100 may generate microcurrent simultaneously, or parts of the contact electrodes 147 in the breast-pumping care accessory 100 generate microcurrents. The microcurrent generated by the contact electrodes 147 in the breast-pumping care accessory 100 may be the same as or different from each other. This is conducive to meeting the individual needs of different target breast-pumping care modes for massage.

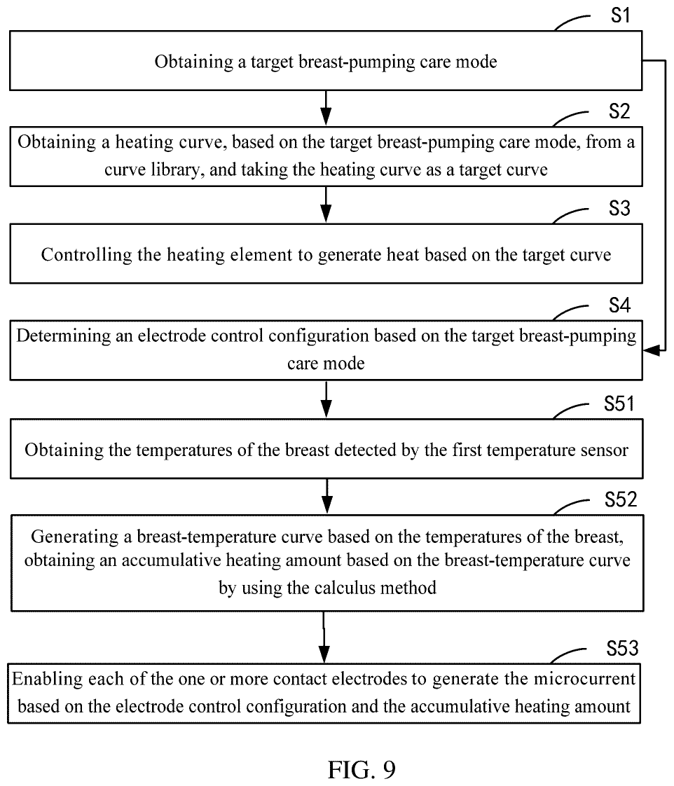

As shown in FIG. 9, in some embodiments, the care assembly 1 may further include a first temperature sensor 125. The first temperature sensor 125 is arranged on the first attaching element 12. The first temperature sensor 125 is configured to detect the temperature of the breast of the living body. The controller 11 is electrically connected to the first temperature sensor 125.

When the care controlling method is executed, in performing the operation of controlling the contact electrode to generate a microcurrent based on the electrode control configuration, the controller 11 is further configured to implement the following operations.

S51, the temperatures of the breast detected by the first temperature sensor 125 may be obtained.

S52, a breast-temperature curve may be generated based on the temperatures of the breast, an accumulative heating amount may be obtained based on the breast-temperature curve by using the calculus method.

S53, each of the one or more contact electrodes 147 are controlled or enabled to generate the microcurrent based on the electrode control configuration and the accumulative heating amount.

In some embodiments, a breast-temperature curve may be generated based on the temperatures of the breast, an accumulative heating amount may be obtained based on the breast-temperature curve by using the calculus method, and each of the one or more contact electrodes 147 are controlled or enabled to generate a microcurrent based on the electrode control configuration and the accumulative heating amount. In this way, it is possible to perform a massage based on a combination of the electrode control configuration and the accumulative heating amount, which is conducive to fully satisfying the individual demands of different target breast-pumping care modes for heating and massage.

The first temperature sensor 125 may adopt a temperature sensor. A probe of the first temperature sensor 125 passes through the first attaching element 12 and contacts with the breast of the living body.

The accumulative heating mount is the total amount of heat generated from an acquisition time point of the target breast-pumping care mode to a time point at which the accumulative heating amount is calculated.

In some embodiments, the electrode control configuration describes the correspondence among the generation time point of the microcurrent, the duration of a single massage, the standard amount of heat, and the standard amount of current.

By continuously performing the operations S51 to S52, the accumulative heating amount may be obtained.

In some embodiments, when the accumulative heating amount exceeds the standard heating amount in the electrode control configuration, and a time interval between a current time point and the acquisition time point of the target breast-pumping care mode is greater than the time interval between the generation time point of the microcurrent and the acquisition time of the target breast-pumping care mode, the contact electrode 147 is controlled to generate a microcurrent based on the standard amount of current, and a duration for generating the microcurrent has the same length as a duration of a single massage.

In some embodiments, the electrode control configuration describes the correspondence among an electrode identification, a generation time point of the microcurrent, a duration of a single massage, the standard amount of heat, and the standard amount of current. The electrode identification may be the number of the contact electrode 147 and is configured to uniquely identify one contact electrode 147. This allows independent control of each contact electrode 147, which helps to further improve the massage effect.

In some embodiments, the care assembly 1 further includes a second temperature sensor 127. The second temperature sensor 127 is configured to detect the ambient temperature of the living body. The controller 11 is electrically connected to the second temperature sensor 127.

When the care controlling method is executed, in the operation of controlling the heating element 14 to generate heat based on the target curve, the controller 11 is further configured to implement the following operation.

S31, the ambient temperature detected by the second temperature sensor 127 may be obtained.

S32, the target curve may be corrected based on the ambient temperature.

S33, the heating element 14 may be controlled or enabled to generate heat based on the corrected target curve.

In some embodiments, the heating element 14 is controlled to generate heat based on the target curve which is corrected based on the ambient temperature, such that the breast-pumping care accessory 100 according to some embodiments of the present disclosure is suitable for living bodies at different ambient temperatures, which is conducive to ensuring the care effect.

The second temperature sensor 127 may adopt a temperature sensor. A probe of the second temperature sensor is in contact with the ambient environment.

For the operation S32, the target curve is corrected based on the ambient temperature, the correction of the target curve is a correction to the temperature in the target curve, and/or, a correction to the time point in the target curve. In some embodiments, based on the ambient temperature, correction configuration is determined by the look-up table method, and the target curve is corrected through the correction configuration. The correction configuration is data that describes the correspondence among a temperature range, time correction data, and temperature correction data. The time correction data is configured to describe a correction operation, a magnitude of the correction, or the numerical value of the time point. The temperature correction data is configured to describe the correction operation, the magnitude of the correction, or the numerical value of the temperature. The correction operation may be one or more of increasing, decreasing, or keeping unchanged.

In some embodiments, the breast-pumping care accessory 100 further includes a sound reminder 16. The controller 11 is electrically connected to the sound reminder 16.

When the care controlling method is executed, after the operation of controlling the heating element 14 to generate heat based on the target curve, the controller 11 may be further configured to implement the following operations: obtaining a heating completion signal; in response to the heating completion signal, controlling the sound reminder 16 to play a prompt sound to remind the completion of the care.

In some embodiments, the prompt sound configured to remind the completion of the care is played through the sound reminder 16, which may remind the user to stop the hot compress in time without needing to actively check the progress of the hot compress, and the user experience may be improved in case that the effect of the hot compress is ensured.

The sound reminder 16 may be implemented as a buzzer.

The heating completion signal is a signal indicating that the heating is completed. Based on the target curve, the heating element 14 is controlled or enabled to generate heat. When the heating is completed, the heating completion signal is triggered.

The prompt sound configured to remind the completion of the care is a signal to remind that the care is completed or finished.

The above embodiments are only used to illustrate the technical solution of the present disclosure and are not used to limit the present disclosure. Although the present disclosure is described in detail with reference to the foregoing embodiments, those skilled in the art should understand that the technical solutions disclosed in the foregoing embodiments may also be modified, or some of the technical features may be replaced or substituted. These modifications or substitutions do not make the essence of the corresponding technical solutions departed from the spirit and scope of the technical solutions of various embodiments of the present disclosure, and shall be included in the scope of protection of the present disclosure.

What is claimed is:

1. A breast-pumping care accessory, comprising: a care assembly, the care assembly comprising:
a first attaching element, configured to be attached to a breast of a living body when the breast-pumping care accessory is in operation;
a second attaching element;
a heating element, located between the first attaching element and the second attaching element, wherein the first attaching element is attached to the second attaching element to fix the heating element;
a controller, electrically connected to the heating element; and
an energy supply element, configured to supply electric energy to the heating element and the controller; and
wherein the controller is configured to perform a care controlling method, and the method comprises:
obtaining a target breast-pumping care mode;
obtaining a heating curve, based on the target breast-pumping care mode, from a curve library, and taking the heating curve as a target curve; and
controlling the heating element to generate heat based on the target curve, wherein the target breast-pumping care mode comprises any one of a pre-pumping care mode, an in-pumping care mode, and a post-pumping care mode.

2. The breast-pumping care accessory as claimed in claim 1, wherein the second attaching element comprises a mounting portion and a fixing portion, the mounting portion is connected to the fixing portion, the mounting portion is configured to mount the heating element, and the fixing portion is configured to fix the care assembly on a breast pump.

3. The breast-pumping care accessory as claimed in claim 1, further comprising a wearable strap, wherein the wearable strap is connected to an upper end and a lower end of the care assembly, or the wearable strap is connected to a left end and a right end of the care assembly; wherein when the breast-pumping care accessory is in operation, the wearable strap is configured to fix the care assembly to the breast of the living body, such that the first attaching element capable of being attached to the breast of the living body.

4. The breast-pumping care accessory as claimed in claim 1, wherein the care assembly is provided with at least one ventilation through hole; the at least one ventilation through hole penetrates the first attaching element and the second attaching element, or the at least one ventilation through hole penetrates the first attaching element, the heating element, and the second attaching element.

5. The breast-pumping care accessory as claimed in claim 1, wherein the first attaching element and the second attaching element are made of elastic silicon;
the heating element comprises a flexible membrane and a printed circuit, the printed circuit is located on the flexible membrane, and the controller is electrically connected to the printed circuit; and
wherein the flexible membrane comprises a plurality of flexible pieces, each of the plurality of flexible pieces is fan-shaped and comprises a first arc portion and a second arc portion, an arc length of the first arc portion is less than an arc length of the second arc portion, the plurality of flexible pieces are arranged in a ring and distributed at intervals, and the first arc portions of every adjacent two of the plurality of flexible pieces are connected to each other; and
wherein when the breast-pumping care accessory is in operation, the first arc portion is located between a nipple of the breast of the living body and the second arc portion.

6. The breast breast-pumping care accessory as claimed in claim 5, wherein the heating element further comprises one or more contact electrodes, the one or more contact electrodes are electrically connected to the printed circuit, the first attaching element defines one or more electrode through holes, the one or more contact electrodes and the one or more electrode through holes are in one-to-one correspondence, the one or more contact electrodes pass through the one or more electrode through holes correspondingly, and the one or more contact electrodes are configured to provide microcurrent stimulation to the breast of the living body.

7. The breast breast-pumping care accessory as claimed in claim 6, wherein when the care control method is executed, the controller is further configured to implement operations comprising:
determining an electrode control configuration based on the target breast-pumping care mode; and
enabling each of the one or more contact electrodes to generate a microcurrent based on the electrode control configuration.

8. The breast breast-pumping care accessory as claimed in claim 7, wherein the care assembly further comprises a first temperature sensor, the first temperature sensor is arranged on the first attaching element, and the first temperature sensor is configured to detect a temperature of the breast of the living body, and the controller is electrically connected to the first temperature sensor;
wherein when the care control method is executed, the controller is further configured to implement operations comprising:
obtaining the temperatures of the breast detected by the first temperature sensor;
generating a breast-temperature curve based on the temperatures of the breast, obtaining an accumulative heating amount based on the breast-temperature curve by using the calculus method; and
enabling each of the one or more contact electrodes to generate the microcurrent based on the electrode control configuration and the accumulative heating amount.

9. The breast breast-pumping care accessory as claimed in claim 1, wherein the care assembly further comprises a second temperature sensor, the second temperature sensor is configured to detect an ambient temperature of the living body, and the controller is electrically connected to the second temperature sensor;
wherein when the care controlling method is executed, in the operation of controlling the heating element to generate heat base on the target curve, the controller is further configured to implement operations comprising:
obtaining the ambient temperature detected by the second temperature sensor;
correcting the target curve based on the ambient temperature; and
enabling the heating element to generate heat based on the corrected target curve.

10. The breast breast-pumping care accessory as claimed in claim 1, wherein the breast breast-pumping care accessory further comprises a sound reminder, and the controller is electrically connected to the sound reminder;
wherein when the care controlling method is executed, after the operation of controlling the heating element to generate heat based on the target curve, the controller is further configured to implement operations comprising:
obtaining a heating completion signal; and
in response to the heating completion signal, controlling the sound reminder to play a prompt sound to remind the completion of the care.

11. A care controlling method for a breast-pumping care accessory, the breast-pumping care accessory comprising:
a first attaching element, configured to be attached to a breast of a living body when the breast-pumping care accessory is in operation;
a second attaching element;
a heating element, located between the first attaching element and the second attaching element, wherein the first attaching element is attached to the second attaching element to fix the heating element;
a controller, electrically connected to the heating element and configured to implement the care controlling method; and
an energy supply element, configured to supply electric energy to the heating element and the controller; and
wherein the care controlling method comprises:
obtaining a target breast-pumping care mode;
obtaining a heating curve, based on the target breast-pumping care mode, from a curve library, and taking the heating curve as a target curve; and
controlling the heating element to generate heat based on the target curve, wherein the target breast-pumping care mode comprises any one of a pre-pumping care mode, an in-pumping care mode, and a post-pumping care mode.

12. The method as claimed in claim 11, wherein the heating element comprises:
a printed circuit electrically connected to the controller; and
one or more contact electrodes, electrically connected to the printed circuit, wherein the first attaching element defines one or more electrode through holes, the one or more contact electrodes and the one or more electrode through holes are in one-to-one correspondence, the one or more contact electrodes pass through the one or more electrode through holes correspondingly, and the one or more contact electrodes are configured to provide microcurrent stimulation to the breast of the living body.

13. The method as claimed in claim 12, further comprising:
determining an electrode control configuration based on the target breast-pumping care mode; and
enabling each of the one or more contact electrodes to generate a microcurrent based on the electrode control configuration.

14. The method as claimed in claim 13, wherein the care assembly further comprises a first temperature sensor, the first temperature sensor is arranged on the first attaching element, and the first temperature sensor is configured to detect a temperature of the breast of the living body, and the controller is electrically connected to the first temperature sensor;
wherein the method further comprises:
obtaining the temperatures of the breast detected by the first temperature sensor;
generating a breast-temperature curve based on the temperatures of the breast, obtaining an accumulative heating amount based on the breast-temperature curve by using the calculus method; and
enabling each of the one or more contact electrodes to generate the microcurrent based on the electrode control configuration and the accumulative heating amount.

15. The method as claimed in claim 11, wherein the care assembly further comprises a second temperature sensor, the second temperature sensor is configured to detect an ambient temperature of the living body, and the controller is electrically connected to the second temperature sensor;
wherein the controlling the heating element to generate heat base on the target curve further comprises:
obtaining the ambient temperature detected by the second temperature sensor;
correcting the target curve based on the ambient temperature; and
enabling the heating element to generate heat based on the corrected target curve.

16. The method as claimed in claim 1, wherein the breast breast-pumping care accessory further comprises a sound reminder, and the controller is electrically connected to the sound reminder;
wherein after the controlling the heating element to generate heat based on the target curve, the method further comprises:
obtaining a heating completion signal; and
in response to the heating completion signal, controlling the sound reminder to play a prompt sound to remind the completion of the care.
